(19) United States
(12) Patent Application Publication
(10) Pub. No.: US 2019/0175939 A1
(43) Pub. Date: Jun. 13, 2019

(54) URINARY RADIATION SENSOR CATHETER

(71) Applicant: AngioDynamics, Inc., Latham, NY (US)

(72) Inventors: John ISHAM, Houston, TX (US); Tamsen VALOIR, Houston, TX (US); Steven C. LEPKE, Wakefield, MA (US); Eric HYMAN, Arlington, TX (US)

(73) Assignee: AngioDynamics, Inc., Latham, NY (US)

(21) Appl. No.: 16/259,336

(22) Filed: Jan. 28, 2019

Related U.S. Application Data
(63) Continuation of application No. 14/881,023, filed on Oct. 12, 2015, now abandoned.
Application No. 14/881,023 is a continuation-in-part of application No. 14/470,707, filed on Aug. 27, 2014, now Pat. No. 8,953,912.
Application No. 14/470,707 is a division of application No. 13/444,584, filed on Apr. 11, 2012, now Pat. No. 8,885,986.
(60) Provisional application No. 62/063,196, filed on Oct. 13, 2014.
(60) Provisional application No. 61/481,503, filed on May 2, 2011.

(51) Int. Cl. A61N 5/10

(52) U.S. Cl. CPC A61N 5/1014; A61N 5/1067

(57) ABSTRACT

A simple method of making robust radiation sensor cables using a special fiber cap that holds a scintillating fiber therein directly abutting an end of a fiber optic cable, thus providing a clean and protected connection therebetween.

### CROSS-REFERENCE TO RELATED APPLICATIONS

This application is a Continuation-In-Part to U.S. Patent Application 61/481,503, filed May 2, 2011, U.S. patent application Ser. No. 13/444,584 (U.S. Pat. No. 8,885,986), filed Apr. 11, 2012, and U.S. patent application Ser. No. 14/470,707 (U.S. Pat. No. 8,953,912), filed Aug. 27, 2014. It also claims priority to 62/063,196, filed Oct. 13, 2014. Each of these is incorporated by reference in its entirety for all purposes herein.

### FIELD OF THE DISCLOSURE

This invention relates to radiation sensor cables of very small diameter, such that they are suitable for use in medical applications, and in particular the tiny cables can be fitted into urethral and other small diameter catheters.

### BACKGROUND OF THE DISCLOSURE

A scintillator is a special material that exhibits scintillation—the property of luminescence when excited by ionizing radiation. Luminescent materials, when struck by an incoming particle, absorb its energy and scintillate, in other words they reemit the absorbed energy in the form of light.

A scintillation detector or scintillation counter is obtained when a scintillator is coupled to a light sensor such as a photomultiplier tube (PMT), photodiode, PIN diode or CCD-based photodetector. The light sensor will absorb the light emitted by the scintillator and reemit it in the form of electrons via the photoelectric effect. The subsequent multiplication of those electrons (sometimes called photo-electrons) results in an electrical pulse that can be analyzed and provides meaningful information about the particle that originally struck the scintillator. In this way, the original amount of absorbed energy can be detected or counted.

The term “plastic scintillator” typically refers to a scintillating material where the primary fluorescent emitter, called a fluor, is suspended in a solid polymer matrix. While this combination is typically accomplished through the dissolution of the fluor prior to bulk polymerization, the fluor is sometimes associated with the polymer directly, either covalently or through coordination, as is the case with many Li₆ plastic scintillators. Polyethylene naphthalate has been found to scintillate without any additives and is expected to replace existing plastic scintillators due to its higher performance and lower price.

The advantages of plastic scintillators include fairly high light output and a relatively quick signal, with a decay time between 2-4 nanoseconds. The biggest advantage of plastic scintillators, though, is their ability to be shaped, through the use of molds or other means, into almost any desired form with a high degree of durability.

In the field of medical radiation therapy, plastic scintillation detectors are used to convert radiation energy into light energy, and the light photons are counted to accurately determine the radiation dose. The scintillating plastic must transfer its photons to a device that can read them, which is commonly done by coupling one or more scintillating fibers to one or more plastic optical fibers (POF). The POF is then connected to a device that can read and analyze the optical output.

This type of sensor is an “active” radiation sensor, not a “passive” radiation sensor. A passive detector is a device that is used to measure levels of ionizing radiation exposure but not in real time. Example includes the Film Badge and the Thermo Luminescent Dosimeter (TLD). The detectors are passive because they need to be “read” at a later stage in order to ascertain the level of exposure recorded. An “active” detector, by contrast, can provide real time information. The sensor described herein gives near real time dosage information, and the delay is much shorter that other active sensors because of the relatively quick signal, with a decay time between 2-4 nanoseconds.

Manufacturing a high volume of such sensor cables is extremely difficult because an accurate and repeatable connection of the plastic scintillator fiber to the plastic optical fiber is required. The problem arises from working with small diameter optical fibers that must be constructed accurately, yet at a low cost and with good durability, reliability and sensitivity.

The current process used to create a sensor cable with a plastic scintillation detector relies on many precise, time-consuming steps, and such cables have thus not reached mainstream use due to the cost of manufacture. First, both ends of the scintillating fiber must be cut and polished. These cuts and polishes are difficult to do because the diameter (1 mm) and length (2 mm) of the scintillation fiber are very small. Next, the optical fiber must be cut, stripped and polished. Then the scintillating fiber is attached to the optical fiber with optical adhesive. A small piece of the optical fiber's jacket can be used to hold the two fibers in place when adhering. This step is challenging due to the small size of the fibers and the need to perfectly align their cores. A black paint or coating is then applied to the distal end of the fiber in order to keep the assembly light tight. The finished assembly is vulnerable to breakage because reliance is placed on the strength of the epoxy bond to hold the assembly together, and on a soft jacket material (PE or PVC) to hold the assembly in alignment. Due to the labor intensive process and time consuming steps, it is very expensive to produce a detector in this fashion, and the process also introduces variability from detector to detector. The current process also uses twist on (FC) or screw on (SMA) metal-bodied fiber connectors at the other end of the sensor cable. Applying these connectors adds more time to the process, and the FC and SMA connectors are expensive.

Therefore, a need exists for a novel manufacturing process and system for a radiation sensor cable to solve these problems. Furthermore, there is a need for a sensor that is of sufficiently small diameter to fit into a urinary catheter for measuring radiation dosage in the urethra when, e.g., when irradiating the prostate.

### BRIEF SUMMARY OF THE DISCLOSURE

Generally speaking, the disclosure relates to tiny plastic scintillator detector cables, suitable for use in urinary and other small diameter catheters, methods of fabricating same and various applications therefor. The tiny and inexpensive scintillator detectors are used to assess radiation dosage in real time, and provide a tremendous advance in the field, which heretofore has lacked such tiny, inexpensive detectors for use inside a body cavity at the actual location of the radiation therapy.

Applications include external beam radiation therapy (XRT), stereotactic radiosurgery/stereotactic radiotherapy (SRS/SRT), intensity modulated radiation therapy (IMRT), dynamical arc therapy, tomotherapy treatments, and any similar application where radiation sensing in a small area is needed, including non-medical applications.

In one embodiment, the plastic scintillator detector cable consists of a single, short length of scintillator fiber operably coupled to a suitable length of optic fiber, which has a standard data coupler or connector at the end of the cable opposite the scintillator fiber. The scintillator detector is thus at the distal end of the cable and a suitable data coupler is at the proximal end, and the entirety of the cable is enclosed in a flexible, opaque covering or coating. This sensor is fitted inside a urinary catheter, which preferably includes a urine draining lumen, as well a sensor lumen. The sensor lumen can also include a balloon (e.g., a Foley catheter), provided that the lumen valving is designed to accommodate the cable passing therethrough in a leak proof manner or the ends are bifurcated such that the sensor exit and air inflation means are separate.

In another embodiment, the cable is hardwired directly to a photodetector, thus avoiding connector use. However, a connector is preferred as it allows for quick and easy replacement of damaged cables.

In another embodiment, the cable has at least two separate, but closely juxtaposed, plastic scintillator detectors. The two detectors are parallel, but offset from one another in the longitudinal axis, so that radiation can be simultaneous assessed at two ends of a target, such as on either end of the prostrate or both ends of an irradiated throat area, and the like. However, this embodiment increases the diameter of the cable, and a single detector is preferred for urinary use as providing the smallest possible diameter of ≤3 mm, 2 mm, ≤1 mm or even less. Of course, the connector is excluded from this measurement, as it is much larger, but never enters the body. The diameter of the single sensor cable is approximately 2.8 mm. It uses a 1 mm optical fiber and 1 mm scintillating fiber. The added diameter is due to the fiber jacket, plastic cap and shrink tubing.

In another embodiment, an additional fiber optic cable without plastic scintillator detector can be added thereto, and can serve the function of allowing the subtraction of any background signal, which can arise from the inherent dark current of the PMT or mostly Cerenkov light generated in the fibers. However, these effects are negligible for photon beams, and thus this extra cable is not needed.

Additional plastic scintillation detectors can be added if desired to assess radiation in three or more places along a longitudinal radiation axis. However, single scintillation detectors can also be used where sufficient for the application in question, e.g., where the area to be irradiated is quite small.

Where it is desired to assess radiation levels over more than one axis, e.g., with a larger radiation zone, a second plastic scintillator detector cable can be added, somewhat offset from the first cable (offset in the axis perpendicular to the cable), although this will obviously increase the overall size and cost of the device accordingly.

The scintillator detector can be combined with any medical device suitable for insertion into a body cavity, such as a prostate balloon, vaginal balloon, catheter, needle, brachytherapy—applicator, surgical implements, and the like.

For balloon usage, a small strip of balloon material can be welded to the outer surface thereof, forming a pocket or channel, and the scintillator cable threaded therethrough, thus reliably positioning the detector on the outer surface of the balloon. Alternatively, the cable can be placed inside the balloon and held with one or more spot welds and/or small strips of balloon material or other attachment means.

For solid medical devices, such as brachytherapy applicators, a small tube can be affixed thereto, and the tiny cable threaded inside the small tube, or the cable can be affixed directly to the applicator. Alternatively, a removable balloon can be provided for the applicator, such as is already described. The cable can also be threaded inside a catheter or needle, and other device used to access a body cavity.

The scintillator detector cable has any suitable data connector or adaptor at the proximal end thereof, and is plugged into any existing or dedicated signal detection and computer system for collecting, analyzing and outputting the data collected by the scintillator detector.

Suitable connectors include FDDI, ESCON, SMI, SCRJ, and the like, and will of course vary according to the system that is intended to be used with the scintillator detector cable. The data connectors can be single connectors, even for a dual or triple detector embodiment, but preferably a dual connector is used for the dual detector embodiment, etc., which keeps the cables neat and can prevent plugging sensors into the wrong channels if the connector has asymmetry.

Because the scintillator detector is quite small, novel fabrication methods were developed to allow cost effective, reliable manufacture and assembly therefore. A special cap was therefore designed to allow the scintillator fiber to be reliably connected to the fiber optic cable. This cap is essentially tube shaped with a blind end (a covered or closed end), such that the scintillator fiber fits entirely into the blind end, and the fiber optic cable fits behind it. Thus, the hollow interior closely holds the ends of the two fibers in close juxtaposition (direct contact or “abutting”) without the need for any adhesive between the ends of the two fibers, which greatly improves both sensitivity and reliability. The hollow interior is thus shaped to closely fit the naked fibers, and in many instances will have a circular cross-section, although this can of course vary if the fiber cross section of the cables is varied.

The tube could also have two open ends, but one closed end is preferred as better protecting the fragile fiber, and avoiding a closure step. However, a dual opening cap may be preferred for longer fibers since the dual opening variant can be loaded from either end. The cap can also comprise two components fitted together, e.g., by threadable or snap fits ends, but the unitary construction is the simplest to make and use. Where the tube has an open end, it can be covered with heat shrinkable tubing, a jacket, opaque coating, snap fit lid, or any other means of making it light tight, and preferably water tight. A snap fitting lid can easily be attached to the tube with a small hinge, thus providing a unitary construction that can be made by injection molding, and still allowing tube loading from both ends.

The cap can also be designed with a small extra space left inside for placement of a fiducial marker. In this way, an imaging device, such as a tungsten, gold, barium, carbon or any other radiopaque or reflective pellet can be placed in the tip of the cable and assist in its placement inside the body. Alternatively, the pellet can be placed outside the cap, e.g., on the outer surface or tip thereof. In fact, the cap can be injection-molded with a small snap fit recess into which an imaging pellet can be snap fit, and an optional plug can fitted over the marker if needed.

The blind cap can be affixed to the optical fiber using an optional bead of adhesive at the open end, which will thus only touch the side of the naked optical fiber, or an external clamp can be used, or the blind cap itself can be made of heat shrinkable material for a tight fit. However, we have found that a harder plastic functions best to keep the two cables aligned, and prefer a high impact polystyrene or similar resin for this purpose. Resins may have a hardness of 65 or less, 55 or less, 45 or below, and preferably has 30-40 Shore D.

Alternatively or in addition thereto, an exterior coating of heat shrinkable material can be added thereto for good strength and fit. The shrink tubing covers at least the detector end of the device and protects the detector, while keeping the components together in a tight bundle that remains flexible and can move in all directions. Where the cap is opaque, part of the cap can protrude from the heat shrinkable tubing. The shrink tubing can also cover most or all of the cable, but this will generally not be needed since plastic optical fibers are usually already jacketed, although the heat shrinkable tubing will also function to keep the fibers tightly bundled and thus may be of benefit. A spray on or dip opaque coating or paint is yet another alternative.

Suitable plastics for the blind cap include high impact polystyrene, polybutadiene, acrylonitrile butadiene styrene, polyvinyl chloride, polycarbonate, polyacrylate, polyethylene terephthalate glycol, high density polyethylene, polypropylene, high impact rigid polyvinyl chloride, and polytetrafluoroethylene, and blends and copolymers thereof. Preferred materials are opaque in color to keep the assembly light tight. Alternatively, the cap can be covered in an opaque material and thus plastics with high clarity can be used, such as polycarbonate and polyacrylate.

Also preferred, the blind cap is constructed from a water equivalent plastic so as to not perturb the radiation dosage, and it is known in the art how to assess water equivalence at different energy ranges. Where the plastic is not quite water equivalent, it is known how to apply a scaling factor. See D. Mihailescu and C. Borcia, Water Equivalency Of Some Plastic Materials Used In Electron Dosimetry: A Monte Carlo Investigation, Romanian Reports in Physics, Vol. 58, No. 4, P. 415-425, 2006 (incorporated by reference herein).

A hot knife blade is preferably used for cutting each fiber, thereby eliminating the need for polishing. A soldering iron set to 700° F. may be used with a fine point carbon steel blade having a thickness of 0.0235 inches (0.06 cm). Other hot knifes, temperatures, and blade thicknesses are also contemplated, and it is known how to vary the temperature with the material being used. Many industrial hot-knives are available for use, and cutting blocks that function to ensure a 90° cut are also commercially available. Although a hot knife may be preferred, other cutting methods can be used, including laser, water jet, diamond saw, and the like.

Many suitable jacket plastics are known, and preferably are opaque plastics of low antigenicity or medical grade, although any plastic can be used and combined with an appropriate biocompatible coating. Such materials include low smoke zero halogen (LSFH), polyvinyl chloride (PVC), polyethylene (PE), polyurethane (PUR), polybutylene terephthalate (PBT), polyamide (PA), and the like.

Particularly preferred jacket materials are medical grade polyurethanes due to their lack of plasticizers and which are available in a variety of hardness, ranging from 60 Shore D to 90 Shore A. Particularly preferred are softer plastics of 70-80 Shore A and which give the cable considerably flexibility combined with sufficient strength. However, the polyurethane may need to overlay an opaque plastic, such as black PVC, unless opaque pigments are added thereto or an opaque paint is applied thereto.

Also preferred are cable materials that withstand sterilization procedures, such as autoclaving, gamma irradiation or chemical treatments, although sterilization may be optional if combined with a separately sterilizable balloon that can completely contain the sensor, or if a non-sterile device is needed, e.g., for rectal applications.

The invention can be any of the following embodiments, in any combination thereof:

An active radiation sensing urinary catheter, comprising:
a. a urinary catheter comprising an elongated thin tube of less than 15 mm outer diameter
and having a distal end and a proximal end and having first and second lumens therein;
b. said distal end having a rounded, closed distal tip and a urinary port proximal to said
distal tip;
c. said proximal end being bifurcated to make a first end and a second end;
d. said first lumen providing a passageway from said urinary port to said first end;
e. said second lumen having a closed distal end near said urinary port and providing a
passageway from said closed distal end to said second end; and,
f. said second lumen housing a light opaque radiation sensor cable of less than 3 mm
outer diameter, said radiation sensor cable comprising:
i. a plastic scintillating fiber directly abutting a fiber optic cable without adhesive
therebetween;
ii. a proximal end terminating in a connector for a separate detector unit; and
iii. at least one fiducial marker thereon or therein,
g. said radiation sensor cable providing real time (within one second) radiation dosage
information when in use.
A radiation sensing urinary catheter, further comprising a fiber cap being a plastic tube with a
closed and an open end that houses said plastic scintillating fiber and a portion of said fiber optic
cable.
A radiation sensing urinary catheter, wherein said fiducial marker is on a distal tip of said fiber
cap.
A radiation sensing urinary catheter, wherein said fiducial marker is inside a distal tip of said fiber
cap.
A radiation sensing urinary catheter, wherein said radiation sensor cable is ≤2 mm in diameter.
A radiation sensing urinary catheter, wherein said fiber cap is made from a hard polymer of
durometer less than 65 Shore D.
A radiation sensing urinary catheter, wherein said second lumen connects at a distal end to an
exterior balloon surrounding said tube and wherein said second end comprises an inflation valve.
A radiation sensing urinary catheter, a third lumen is inside said elongated thin tube connects at a
distal end to an exterior balloon surrounding said tube and wherein a third proximal end of said
third lumen comprises an inflation valve.
A radiation sensing urinary catheter, wherein said second lumen connects at a distal end to an
exterior balloon surrounding said tube, and wherein said second end is bifurcated to provide a
third end having a one way check valve therein, said second lumen being airtight.
A radiation sensing urinary catheter, wherein said distal tip is a Tiemann tip.
A radiation sensing urinary catheter, further comprising an airtight locking hub having a bifurcated
end opposite a single end, said bifurcated end having an airtight air entry portal and a cable entry
portal, said cable entry portal comprising a cable lock for locking said cable radiation sensor cable
in place inside said urinary catheter, said opposite end having an airtight catheter entry for
receiving said catheter tube.
A radiation sensing urinary catheter, further comprising an airtight locking hub having a bifurcated
end opposite a single end, said bifurcated end having an airtight air entry portal and a cable entry
portal, said cable entry portal comprising means for locking said cable in place, said single end
having means for connecting to said tube in an airtight manner.
A radiation sensing urinary catheter, comprising:
a. a urinary catheter comprising an elongated thin tube of diameter <15 mm having a distal
end and a proximal end and having a first lumen and a second lumen therein;
b. said distal end having a rounded, closed distal tip and a urinary port proximal to said
distal tip;
c. said distal end having a toroidal balloon circumnavigating said tube proximal to said distal
tip;
d. said proximal end being trifurcated to make a first end and a second end and a third end;
e. said first lumen providing a passageway from said urinary port to said first end;
f. said second lumen having an exit port for said balloon and said second lumen providing a
passageway from said balloon to said second end;
g. said second lumen housing a light opaque radiation sensor cable of diameter <3 mm,
said radiation sensor cable comprising:
i. a plastic scintillating fiber directly abutting a fiber optic cable;
ii. a plastic fiber cap being a tube having a closed end tube and an open end, said fiber cap
housing said plastic scintillating fiber at said closed end and a portion of said fiber optic cable;
iii. a proximal end terminating in a connector for a separate detector unit; and
iv. at least one fiducial marker thereon or therein; and,
h. said second end terminating in a one way inflation valve and said third end providing an
airtight exit for said radiation sensor cable, such that said connector lies proximal to said airtight
exit.
A radiation sensing urinary catheter, comprising:
a. a urinary catheter comprising an elongated thin tube having a distal end and a proximal
end and having a first lumen and a second lumen therein;
b. said distal end having a rounded, closed distal tip and a balloon proximal to said closed
distal tip and surrounding said tube such that said balloon has a toroidal shape when inflated,
c. a locking hub at said proximal end of said tube, said locking hub having a bifurcated end
opposite a single end,
d. said single end sized to receive said proximal end of said tube in an airtight manner,
e. said bifurcated end having a first fluid inlet port fluidly connected to said first lumen;
f. said bifurcated end having a second cable entry port with means for locking a position of
said cable therein,
g. said locking hub having means for airtight fluid entry into said first lumen;
h. said first lumen providing an airtight passageway from said first fluid inlet port to said
balloon;
i. said second lumen housing a light opaque radiation sensor cable of less than 2 mm
diameter, said radiation sensor cable comprising:
i. a plastic scintillating fiber directly abutting a fiber optic cable;
ii. a proximal end protruding from said cable entry port and terminating in a connector for a
separate detector unit; and
iii. at least one fiducial marker at or near a distal end of said radiation sensor cable.
A urinary catheter having a radiation sensor cable,
a. said urinary catheter being of diameter less than 15 mm, or less than 12 mm, and having
a balloon circumnavigating said catheter near a distal end thereof and means for inflating said
balloon,
b. said catheter further comprising a light opaque active radiation sensor cable of less than
2 mm diameter, said radiation sensor cable comprising:
i. a plastic scintillating fiber directly abutting a fiber optic cable terminating in a connector
for a separate detector unit; and
ii. at least one fiducial marker at or near a distal end of said radiation sensor cable,
c. said radiation sensor cable capable of real-time (<1 second) dosage measurement when
in use.
A urinary catheter having a radiation sensor cable therein,
a. said urinary catheter comprising a tube of diameter less than 15 mm and having a
balloon circumnavigating said tube near a distal end thereof;
b. said tube having an inflation lumen and a cable lumen therein, said inflation lumen fluidly
connected to said balloon;
c. an adaptor at a proximal end of said tube, said adaptor having a single end for receiving
said tube in an airtight manner, said single end opposite a bifurcated end having a fluid intake
end fluidly connected to said inflation lumen, and a cable entry end fluidly connected to said cable
lumen;
d. said fluid intake end comprising means for inflating said balloon, and said cable entry end
comprising means for locking a cable in position therein;
e. said catheter further comprising a light opaque active radiation sensor cable of less than
2 mm diameter, comprising:
i. a plastic scintillating fiber directly abutting a fiber optic cable terminating in a connector
for a separate detector unit; and
ii. at least one fiducial marker at or near a distal end of said radiation sensor cable,
iii. said radiation sensor cable entering said cable lumen via said cable entry end such that
said connector is proximal to said cable entry end,
f. said radiation sensor cable capable of real-time (<1 second) dosage measurement when
in use.
A method of prostate radiation treatment, comprising:
a. inserting the radiation sensing urinary catheter into the urethra of a patient with prostate
disease;
b. imaging said fiducial marker;
c. adjusting a position of said radiation sensor cable or said radiation sensing urinary
catheter to position said fiducial marker at or near a target area to be treated;
d. applying radiation to said target area;
e. measuring an amount of radiation delivered to said radiation sensor cable;
f. collecting urine throughout said treatment; and,
g. adjusting application of radiation to said target area based on said measured radiation.
A method of prostate radiation treatment, comprising:
a. inserting the radiation sensing urinary catheter into the urethra of a patient with prostate
disease;
b. inflating said balloon with fluid to prevent egress of said catheter;
c. imaging said fiducial marker;
d. adjusting a position of said radiation sensor cable to position said fiducial marker at or
near a target area to be treated;
e. locking said locking hub;
f. applying radiation to said target area;
g. measuring an amount of radiation delivered to said radiation sensor cable; and,
h. adjusting application of radiation to said target area based on said measured radiation.
The method, further including a step of transferring said measured radiation to a medical record
for said patient.
A method, further comprising adjusting a position of said radiation sensor cable to be adjacent a
prostate.
A method, further comprising adjusting a position of said radiation sensor cable to be adjacent a
penile bulb.
A radiation sensor cable, comprising:
a. a distal fiber cap having a tubular shape, hollow interior and a closed end and an open
end, and being made from a hard polymer of durometer less than 45 Shore D,
b. a plastic optical fiber;
c. a plastic scintillation fiber;
d. wherein said plastic scintillation fiber fits completely inside said distal fiber cap at said
closed end and is directly abutted against said plastic optical fiber which partially fits inside said
distal fiber cap and partially protrudes therefrom;
e. an opaque jacket enclosing at least a portion of said distal fiber cap and said plastic
optical fiber; and
f. a proximal adaptor operably connected to a proximal end of said plastic optical fiber;
wherein the maximum diameter of said radiation sensor cable is less than 2 mm (excluding said
proximal adaptor).
A radiation sensor cable, wherein said distal fiber cap is housed inside a lumen of a urinary
catheter having a balloon near a distal end thereof and wherein said proximal adaptor is proximal
to said lumen, said urinary catheter having means for inflating said balloon.
A radiation sensor cable, wherein said balloon urinary catheter comprises:
a. a tube having two lumens therein, an inflation lumen fluidly connected to said balloon and
a cable lumen housing said cable,
b. a proximal adaptor at a proximal end of said tube, said adaptor having a single end for
receiving said tube in an airtight manner, said single end opposite a bifurcated end having a fluid
intake end fluidly connected to said inflation lumen, and a cable entry end fluidly connected to
said cable lumen;
c. said fluid intake end comprising means for inflating said balloon, and said cable entry end
comprising means for locking a cable in position therein.
A radiation sensor cable, wherein said balloon urinary catheter comprises:
a. a tube having at least two lumens therein, an inflation lumen fluidly connected to said
balloon and a cable lumen housing said cable,
b. a proximal adaptor at a proximal end of said tube, said adaptor having a single end for
receiving said tube in an airtight manner, said single end opposite a trifurcated end having a first
fluid intake end fluidly connected to said inflation lumen, and a cable entry end fluidly connected
to said cable lumen and a third end for urine egress;
c. said fluid intake end comprising means for inflating said balloon, and said cable entry end
comprising means for locking a cable in position therein.
A radiation sensor cable, wherein said balloon urinary catheter comprises:
a. a tube having three lumens therein, an inflation lumen fluidly connected to said balloon, a
urine lumen with distal port for draining urine, and a cable lumen housing said cable,
b. a proximal adaptor at a proximal end of said tube, said adaptor having a single end for
receiving said tube in an airtight manner, said single end opposite a trifurcated end having a first
fluid intake end fluidly connected to said inflation lumen, and a cable entry end fluidly connected
to said cable lumen and a third urine drainage end for fluidly connecting to said urine lumen;
c. said fluid intake end comprising means for inflating said balloon, said cable entry end
comprising means for locking a cable in position therein, and said third urine drainage end having
means for connecting to a urine bag.
A radiation sensor, wherein said fiber cap further comprising a fiducial marker thereon or therein.
A method of radiation treatment, comprising:
a. inserting the balloon urinary catheter into the urethra of a patient with prostate disease
until said balloon reaches a bladder;
b. inflating said balloon in said bladder;
c. imaging said fiducial marker;
d. adjusting a position of said radiation sensor cable or said radiation sensing urinary
catheter to position said fiducial marker at or near a target area to be treated;
e. locking said cable in said position;
f. applying radiation to said target area;
g. measuring an amount of radiation delivered to said radiation sensor cable during said
treatment; and
h. adjusting application of radiation to said target area based on said measured radiation.

As used herein, a “simplex sensor cable” refers to a sensor cable having a single sensor. A “duplex” cable has two sensors, typically offset in location. A “triplex” sensor cable would have three scintillators, and so on.

As used herein, a “lumen” is a long hollow conduit that can be used for fluid flow or for housing a cable. A lumen need not have a circular cross-section.

A “tube” as used herein is a long hollow cylinder, having circular cross-section.

As used herein, “real time” dosage detection occurs within one second of a signal being received by the active radiation sensor.

As used herein, “airtight” and “liquid tight” fits are used interchangeably to refer to a tight seal that prevents the escape fluids, whether air or liquid. The balloons in the present disclosure must be air and fluid tight, and by necessity, the locking hubs and some bifurcated ends.

The use of the word “a” or “an” when used in conjunction with the term “comprising” in the claims or the specification means one or more than one, unless the context dictates otherwise.

The term “about” means the stated value plus or minus the usual margin of error of measurement, or plus or minus 10% if no method of measurement is indicated.

The use of the term “or” in the claims is used to mean “and/or” unless explicitly indicated to refer to alternatives only or if the alternatives are mutually exclusive.

The terms “comprise,” “have,” and “include” (and their variants) are open-ended linking verbs and allow the addition of other elements when used in a claim.

The phrase “consisting of” is a closed linking verb and does not allow the addition of any other elements.

The phrase “consisting essentially of” occupies a middle ground, allowing the addition of non-material elements such as labels, instructions for use, and the like.

### BRIEF DESCRIPTION OF THE DRAWINGS

A better understanding of the invention can be obtained with the following detailed descriptions of the various disclosed embodiments in the drawings, which are given by way of illustration only, and thus are not limiting the invention, and wherein:

FIG. 1A is a perspective view of a partially coiled duplex scintillator cable, with adaptor at the proximal end and exploded scintillator detectors at the distal end.

FIG. 1B is a detail exploded view in area B of FIG. 1A of two exposed duplex optical fibers, two scintillating fibers, two rings of adhesive, two fiber caps, and a heat shrink tubing.

FIG. 1C is a detail view in area C of FIG. 1A showing the adaptor.

FIG. 2A is a plan view of the distal end of the duplex plastic optical fiber of FIG. 1.

FIG. 2B is a cross section of the view in FIG. 2A through lines 2B-2B.

FIG. 3A is an perspective view of the fiber cap of the invention.

FIG. 3B is a plan view of FIG. 3A.

FIG. 3C is a cross-sectional view along line 3C-3C of FIG. 3B.

FIG. 4 is a flow diagram of the assembly process.

FIG. 5 is a perspective view of an open cap with hinged snap fitting lid.

FIG. 6 depicts the use of a balloon urinary catheter in a male patient.

FIG. 7 displays urinary catheter gauges.

FIG. 8 displays a trifurcated urinary catheter design with balloon and radiation sensor cable.

FIG. 9 displays a bifurcated urinary catheter design with radiation sensor cable, but without balloon.

FIG. 10A is an exterior view of a simplex sensor cable.

FIG. 10B is an cross sectional view of a portion of a simplex sensor cable.

FIG. 11A shows a locking hub that allows sensor cable and air entry, and FIG. 11B shows the locking hub in position over the end of the lumen, with the sensor cable protruding therefrom.

FIG. 12 shows a two lumen embodiment that lacks urine drainage features, but shows a locking hub with airtight connectors for air entry and for cable entry. In this embodiment, the locking hub has two fluidic pathways therein, each pathway lining up with the relevant lumen in the catheter tube.

FIG. 13 shows the locking hub in cross section, as well the catheter tube in cross section. In this view the two fluidic pathways of the locking hub can be seen.

### DETAILED DESCRIPTION OF THE DISCLOSURE

The following detailed descriptions are exemplary only and not intended to unduly limit the scope of the claims beyond the plain and ordinary meaning of the terms in the medical art of radiation oncology, and their equivalents.

### Sensor Cables

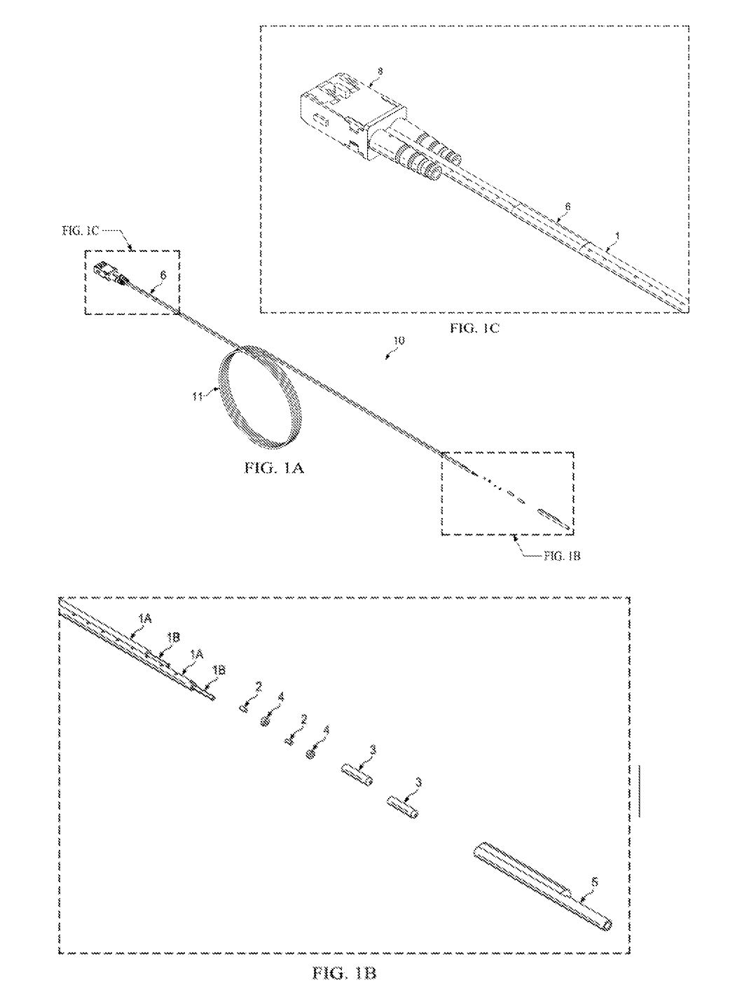

The parts of FIG. 1A-C are listed herein and exemplary materials provided:

| Part No. | Description | Preferred materials |
|---|---|---|
| 1 | Optical fiber | MITSUBISHI SUPER ESKA 1 MM |
| | | DUPLEX PLASTIC OPTICAL |
| | | FIBER SH4002 |
| 2 | Scintillating fiber | BCF-60 SAINT GOBAIN |
| | | SCINTILLATING FIBER PEAK |
| | | EMMISSION 530 NM |
| 3 | fiber cap | HIGH IMPACT POLYSTYRENE |
| 4 | Adhesive | EPOXY TECHNOLOGIES EPO- |
| | | TEK 301 |
| 5 | Heat shrinkable tubing | RAYCHEM THERMOFIT CGPE- |
| | | 105 HEAT-SHRINKABLE TUBING |
| 6 | Lot Code Label | NA |
| 8 | Adaptor | SCRJ CONNECTOR |
| 11 | Coiled section of cable. | NA |
| | Protruding ends allows | |
| | for calibration of each | |
| | cable and is a preferred | |
| | packaging method. | |

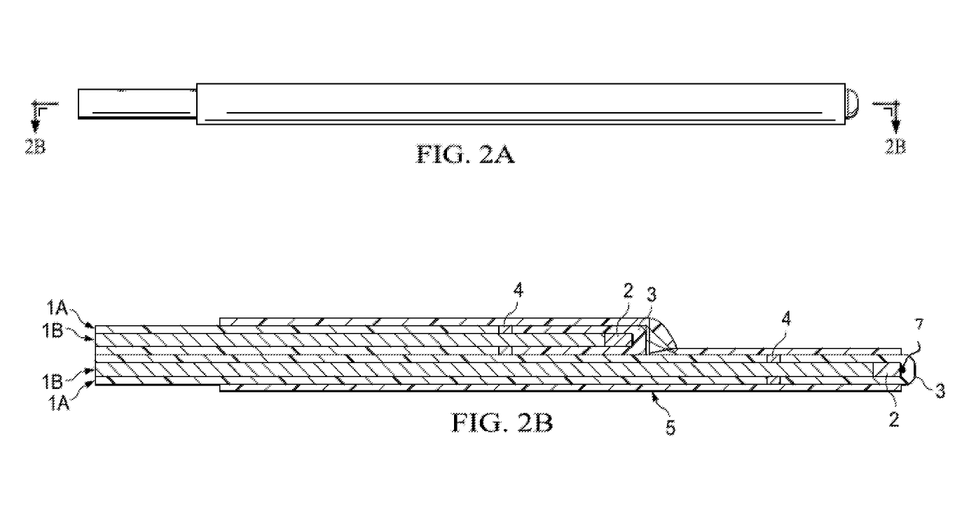

Turning to FIG. 1A-C, the duplex scintillation detector cable 10 has a first and second optical fibers 1. The jacket or covering 1A has been stripped or removed from the portion of the first optical fiber 1 adjacent to the distal ends of each fiber (see also FIG. 2B), leaving a portion of each optical fiber 1B exposed. First and second scintillating fibers 2 are shown, along with drop of adhesive 4 and fiber cap 3. The length of scintillating fibers 2 can be varied, according to needed sensitivity and size of area to be assessed, but typically 1-10 mm of length will suffice. We have used 2-3 mm lengths in prototypes.

The scintillating fibers 2 fit into the fiber caps 3, followed by the naked optic fibers 1B, and a drop of epoxy 4. Heat shrink tubing 5 covers the components, which are shown assembled in FIGS. 2A and 2B. At the far end, an adaptor 8 is found, in this case a dual jack adaptor. Label 6 is also shown, but may be placed anywhere on the cable or even on packaging and is not considered material. There is no adhesive 4 on the abutted ends or faces of the respective scintillating fibers 2 and optical fibers 1B, thus signal is optimized.

The duplex optical fiber 1 may be a Super Eska 1 mm duplex plastic optical fiber SH4002 available from Mitsubishi Rayon Co., Ltd. of Tokyo, Japan, although other duplex optical fibers are also contemplated. Although duplex optical fibers 1 are shown, it is also contemplated that a single optical fiber may be used or additional fibers can be added. Single fiber sensor cables are preferred for urinary catheters, due to the size limitations.

The scintillating fibers 2 may be a BCF-60 scintillating fiber peak emission 530 NM available from SAINT-GOBAIN CERAMICS & PLASTICS™, Inc. of Hiram, Ohio, although other scintillating fibers are also contemplated.

FIG. 2A shows a plan view of the detector end of cable and line 2B-2B, through the center of the cable. FIG. 2B is a cross-section at line 2B-2B. Seen here are scintillator fibers 2, inside cap 3, and immediately distal to naked optical fibers 1B. Heat shrink tubing 5 covers the detector/distal end of the cable, thus making a detector assembly. Tubing 5 is shown with a small amount of the distal-most cap protruding, but placement can vary as long as the bundle is tightly held and opaque. Optical fibers past the cap 3 are covered by jacket 1A. A bead of optional adhesive 4 is placed at the end of cap 3 and does not touch the ends of the fibers, but a small amount can travel by capillary action between optical fiber 1B and the inside of cap 3.

Fiducial marker 7 is shown outside the cap, but can also be inside the cap.

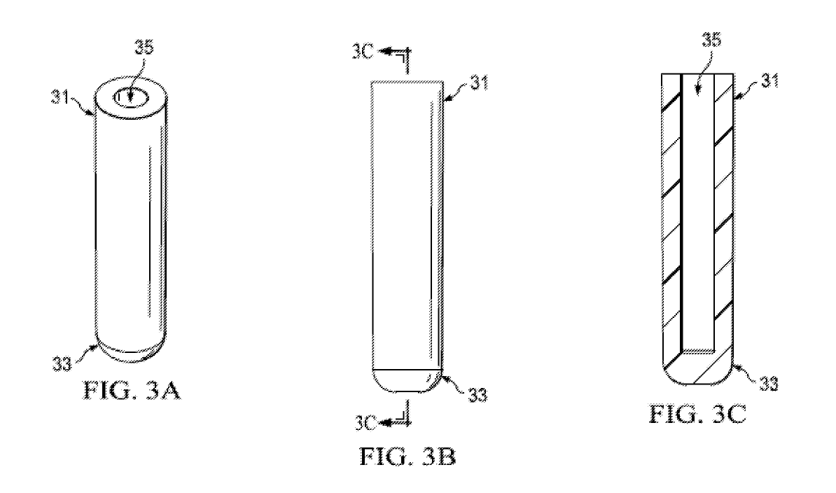

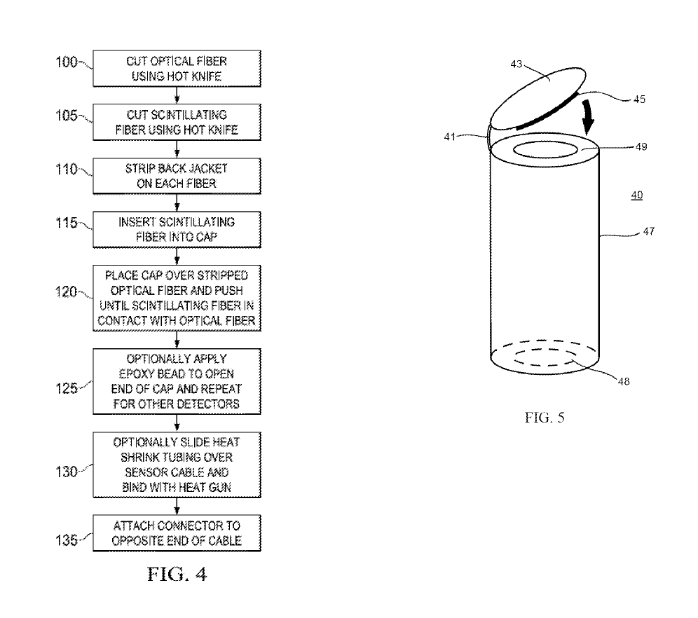

The fiber cap 3 is shown in more detail in FIGS. 3A-C. Cap 3 has an open end 31 and a closed end 33 defining a hollow interior 35 into which fibers 1, 2 tightly fit. The cap 3 is constructed from a water equivalent material, such as polystyrene, and may be opaque in color to keep the assembly light tight. A high impact polystyrene may be used, with a Mold-Tech 11010 texture (2.0 minimum draft, and 0.00100 inch depth) or smoother, although other materials and textures are also contemplated. See e.g., henryplastic.com/PDFs/Mold-Tech %20Tips.pdf, incorporated by reference herein in its entirety for all purposes.

The use of a pair of plastic optical fibers 1 and pair of scintillator fibers 2 allows a dual detector system using two fibers jacketed together to form a single cable. However, the detectors are still independent and give separate measurements of radiation dosage at each location. The duplex scintillator cable 10 combined with the longitudinally offset positioning of two scintillating fiber tips 2 allows for the detection of two distinct areas of radiation in a single sensor cable device. Additional scintillating fibers and optical cables may be added to the cable for additional detection areas.

The small length of shrink tubing 5 covers the detector end of the device and protects the detectors 2 while keeping the assembly together in a tight bundle. The bundle is allowed to flex and move in all directions. If desired, the shrink tubing can cover a longer length of the cable than is shown herein. Paint or coatings can be used, but shrink tubing may be preferred as easy to assemble, and providing some degree of protection, with a perfect fit. However, where size is critical, a paint or other thin coating may be preferred.

The diameter of the cable herein described is very small, and the device is thus tiny enough to be added to existing medical devices for a variety of radiation applications. Preferably, the cable diameter (excluding the proximal adaptor/connector) is less than 5 mm, and preferably less than 4, 3, or 2 mm. Yet, in spite of its small size, the device is robust and easily manufactured. A single sensor cable is preferably 2 mm or less, 1 mm or less, or even as small as 0.3-0.5 mm in diameter.

A hot knife may be used to make the process more efficient. By cutting each optical fiber distal end 1 and each scintillating fiber 2 with the hot knife blade, the polishing step of the past may be eliminated. The hot knife cuts a smooth and uniform fiber surface with no scraping or cracking, producing light transmittance results on par with polished fibers.

The optical adhesive used in the past may also be omitted from the method and system.

Instead of using adhesive between the exposed optical fiber ends, as is done in the prior art, the optical and scintillating fibers are aligned using the fiber cap 3 and secured by applying optional adhesive 4 only to the open end 31 of the cap 3. The bond is between the cap 3 and the exposed sides of the optical fiber 1 and increases the strength of the assembly and reduces the accuracy needed at the adhesive joint, however the adhesive is optional, as is the shrink tubing.

One embodiment of the assembly process is illustrated in FIG. 4, and is as follows.

Step 1: Cut the plastic optical fiber 1 to length using the hot knife at step 100.

Step 2: Cut the plastic scintillation fiber 2 to length using the hot knife system at step 105.

Step 3: Strip back the jackets (if any) on each fiber 1, 2 to a specified length at step 110.

Step 4: Insert the bare scintillation fiber 2 into the scintillating cap 3 at step 115 and gently push until seated at the blind terminus.

Step 5: Insert bare optic fiber 1 into scintillating cap 3. Gently push until the optical fiber 1 is in good contact with the scintillation fiber 2 at step 120.

Step 6: Apply the optional bead of the UV cure epoxy or other adhesive 4 around the open end 31 of the fiber cap 3 where the optical fiber 1 is exposed at step 125. No epoxy 4 contacts the scintillation fiber 2 or the respective abutting ends of the two fibers because only a small amount of adhesive is used.

Step 7: Slide the optional heat shrink tubing 5 over the distal end of the sensor cable 10 so that the edge of the heat shrink 5 is approximately 1 mm away from the distal end of the most distal scintillation cap 3, although it can also completely contain same or more can protrude, as desired. Use a heat gun or oven to shrink down the tubing 5 over the detectors 2 at step 130.

Step 8: Attach an appropriate connector 8 to the proximal end of the cable 10 opposite the detectors 2 at step 135.

The cable is thus fabricated, and can be labeled, packaged, and optionally sterilized, as needed.

The process allows for a much quicker and more accurate assembly than in the past. The cable assembly may be produced in high volumes with excellent repeatability. Variations on the methods are contemplated, and fewer steps are also contemplated.

FIG. 5 shows an open ended cap 40 that can be of unitary construction made by injection molding. The tube 47 has two open ends 48, 49, each of which can accessed for manufacture of the cable. Once the two fibers are in place, lid 43, held with flexible thin hinge 41, snaps shut, with an annular edge or lip 45 serving a snap fit function and making the cap light and water tight.

### Urinary Catheters

Urinary catheters are typically made of latex or silicone rubber, although a trend has been made of using polyurethane or carbothane (a polyurethane/polycarbonate copolymer) rather than silicone because it allows for better catheter strength and softness, while still maintaining a large internal diameter. Silicone rubber catheters are believed to be superior to latex catheters, as silicone is more biocompatible, causes less cell death, less likely to become encrusted, and more resistant to bacterial colonization, although latex is low cost. Silastic catheters have decreased incidences of urethritis and, possibly, urethral stricture and can also be used long-term. Polyvinylchloride and polyethylene catheters have a wide lumen enabling a rapid flow rate, are recommended for short-term post-operative use, but cause greater patient discomfort. Pre-lubricated, sterile non-latex catheters coated with polyvinylpyrrolidone, are water absorbent, cause 90%-95% less urethral friction trauma to the urethra, and are also indicated long-term. Other friction reducing agents can be coated on the catheter exteriors.

The catheter can either have one, two or three lumens therein, but preferably has at least two lumens.

The multiple lumens can either be nested (coaxial) or split and split lumens can be made by extrusion or by the merging of separate lumens with e.g., heat, and combinations thereof are also possible (e.g., U.S. Pat. Nos. 3,769,981, 3,634,924, 3,746,003, 4,793,351, 5,167,623, 7,500,982, each incorporated by reference in its entirety).

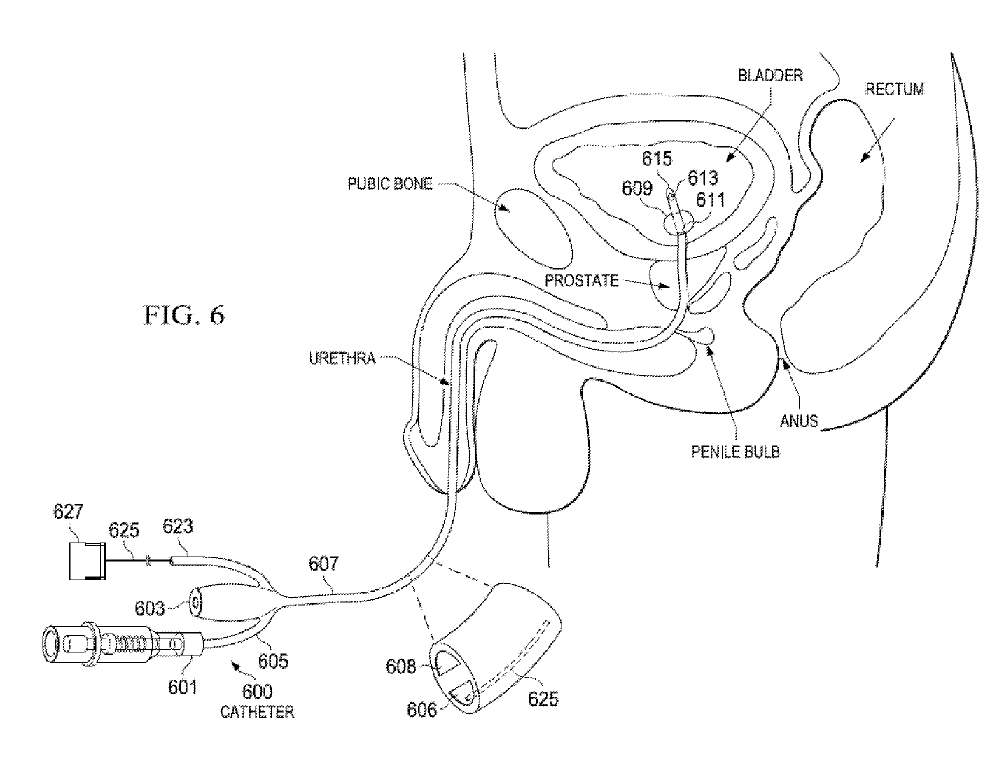

FIG. 6 shows an example of how a balloon catheter is inserted into the male urinary tract. The balloon catheter 600 in this case is trifurcated at the proximal end to provide a inflation valve 601, usually a one way check valve, as well as a urine bag connector 603, which typically is connected by friction fit, and an airtight outlet 623 for sensor cable 625 ending in adaptor 627 (cable length not drawn to scale).

Inflation lumen 605 provides a passage from the inflation valve 601 to the balloon 609, and balloon port 611 allows air or fluid to be pumped into the balloon when situated inside the bladder, thus preventing accidental egress. This same passage also houses the sensor cable 625, although the passageway bifurcates (608/606 lumens) near the proximal end to give an airtight portal 623 for the cable 625 on one branch, and an inflation means 601 on the other branch.

A second passageway is the urine lumen 607, reaching from urine bag connector 603 to urine port 613, which is usually just shy of the distal tip 615, all of which are situated distal to the balloon. Urine can enter the port 613, travel down the urine lumen 607 and exit the connector 603 to a collection bag (not shown). When inflated, balloon 609 prevents the catheter from withdrawing from the bladder.

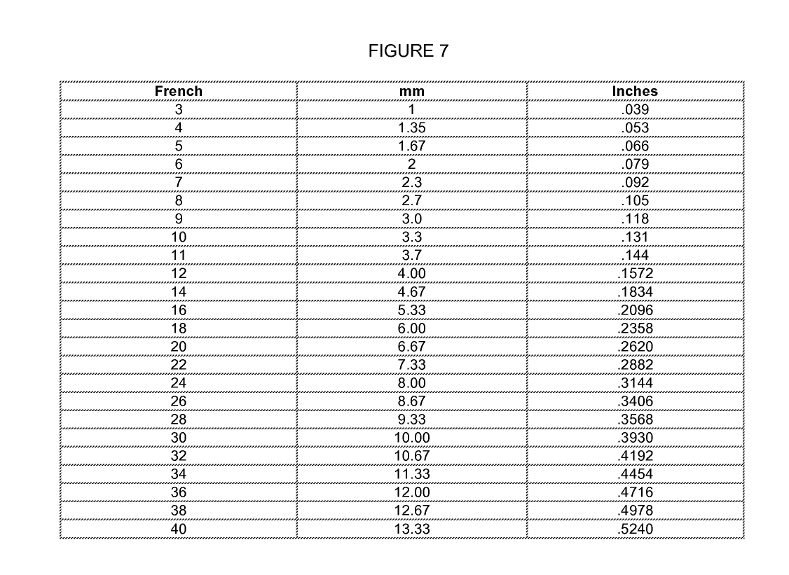

Catheter size is expressed in Charriere (Ch) units, which reflects the catheter's outer diameter in millimeters (1 Ch=0.33 mm diameter) or French units (fr=circumference in millimeters). The smallest size of the catheter consistent with effective drainage is used. In the presence of infection or if post-operative bleeding is expected, a larger bore catheter minimizes catheter obstruction. Most urethral catheters are 41-45 cm. long; a shorter catheter (20-25 cm) is more discreet and comfortable in women. Foley catheters typically vary in size from 12 fr to 30 fr (4 to 10 mm) in diameter, with the standard being 14 fr (4.6 mm). The balloon itself varies in size from 5 cc to 30 cc, depending on the needed use, but is typically less than 10 cc. The balloon can either be filled with sterile water or saline or air, but sterile solutions may be preferred in case of accidental leak. FIG. 7 provides a catheter gauge and conversion units.

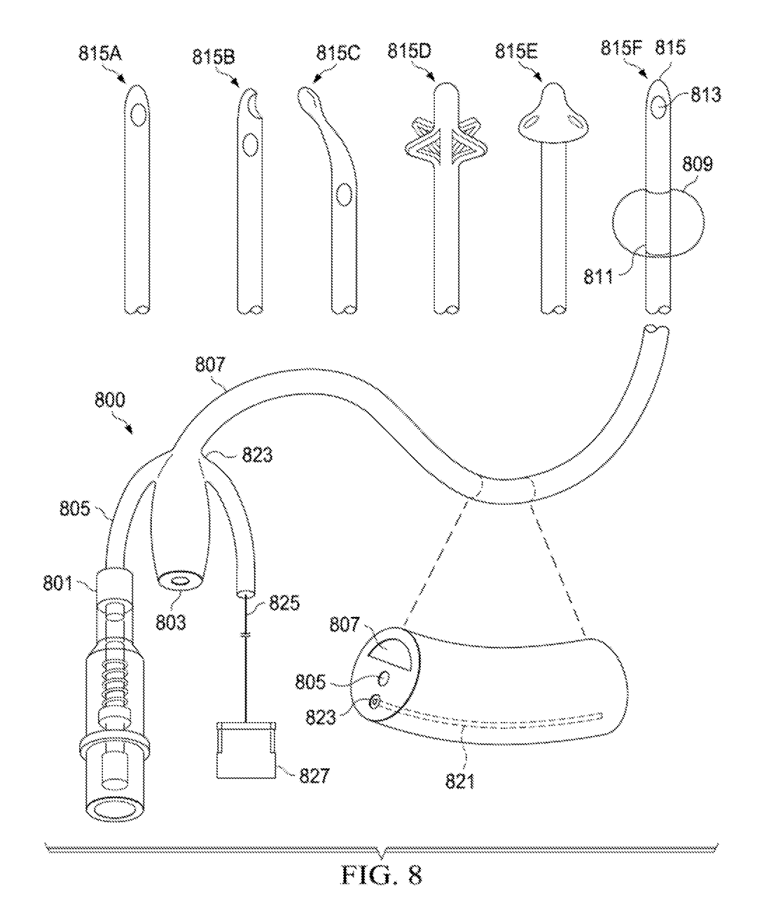

FIG. 8 provides a picture of a balloon catheter equipped with the small diameter radiation sensor cable invented herein. The balloon radiation sensor catheter 800, has a trifurcated proximal end. One end comprises an inflation valve 801, usually a one-way check valve often having luer lock fittings for connection to a syringe. A second end has a urine bag connector 803, and the third end has a sealed outlet 823 for the end 825 of the sensor cable 821, which terminates in an adaptor 827.

In the embodiment of FIG. 8, a separate lumen 823 is provided for the cable in which case the outlet need not be air-tight. However, where the air passageway 805 and cable 821 share the same passageway, the outlet 823 should be air tight, such that air cannot escape the balloon, yet in preferred embodiments will still allow some adjustment of sensor position inside the catheter. A elastomeric seal will provide the necessary seal for outlet 823, yet allow adjustment of cable position therein. Other airtight connectors could be used however.

In other embodiments, the cable is small enough to fit under a strip of adhesive tape on the outside of the catheter (not shown), but this is not preferred as a less robust and less smooth catheter may result. However, with careful choice of materials and a flatter cross section of the cable or groove on the exterior of the catheter, this may be a viable alternative.

Air pockets should be avoided near the sensor itself, as the radiation may travel faster through air, resulting in errors in dosing. A water or tissue equivalent adhesive can be used to fill air pockets, but preferably, the assemble tolerances are such as to minimize air pockets.

In the small cross section of the catheter, can be seen the inflation lumen 805 and the urine lumen 807 and cable lumen 823. The sensor cable 821 can either be positioned in the inflation lumen 805, or an additional lumen 823 can be provided, as shown here.

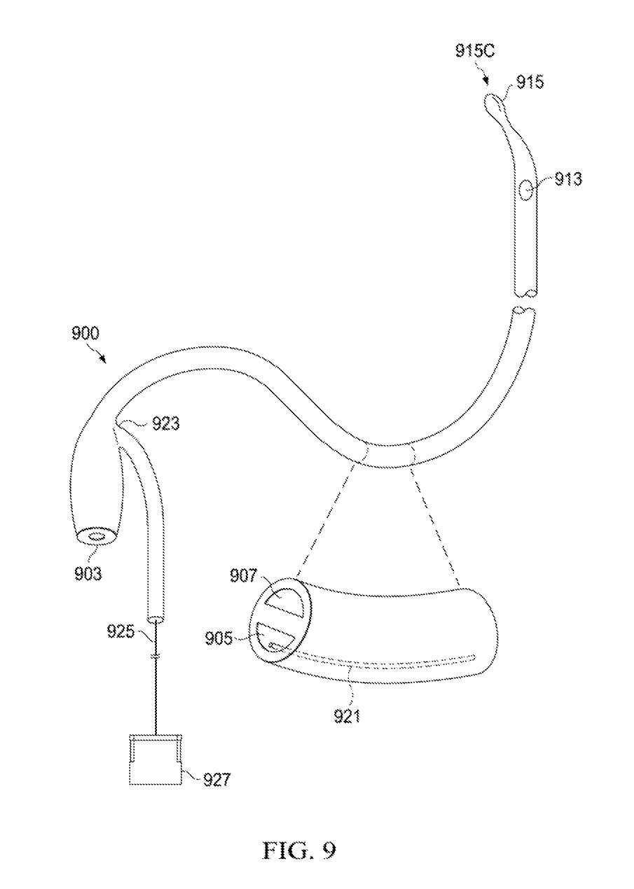

As yet another alternative, the balloon and inflation valve can be omitted and the second lumen dedicated to sensor use, as shown in FIG. 9. Balloon 809, balloon port 811, urine port 813, and distal tip 815 complete the catheter.

Any tip can be used with the urinary catheter of the invention, including 815A-Simple urethral catheter; 815B—Open-ended (whistle-tip) catheter; 815C—Coude Catheter (Tiemann); 815D—Wing-tip (Malecot) catheter; or 815E—Mushroom (de Pezzer) catheter; and 815F—simple urethral catheter. However, the Tiemann tip in 815C may be preferred because it is designed to accommodate the enlarged prostate that occurs with benign and metastatic prostate cancers.

FIG. 9 provides a picture of a non-balloon urinary catheter equipped with the small diameter radiation sensor cable invented herein. The radiation sensor catheter 900, has a bifurcated proximal end. One end comprises a urine bag connector 903, and the second end has an outlet 923 for the end 925 of the sensor cable 921, which terminates in an adaptor 927.

If this embodiment is not to be combined with balloon, then outlet 923 need not be air or liquid tight, and thus the position of the sensor can easily be adjusted along the length of the catheter by pulling or pushing on the cable. However, the outlet should still be tight enough to provide a friction fit so that the cable does not easily move on its own. Alternatively, a locking mechanism (e.g., a clamp or snap fit lock) can be provided to hold the cable in place once adjusted.

In the small cross section of the catheter, can be seen the sensor lumen 905, sensor cable 921 therein and the urine lumen 907. Urine port 913, and e.g., Tiemann tip 915C complete the radiation sensor catheter.

Single or “simplex” sensor cables can be built less than one mm in diameter, indeed as small as 0.3-0.5 mm, and thus can easily fit inside a 4 mm catheter, which is the standard size for urinary use. Such cables easily fit inside urinary (or cardiovascular) catheters in the manner shown herein, and thus provide real-time dosage information on radiation treatment of the genitourinary tract, arteries and veins, and especially the prostate. The urinary sensor cable is as described herein, but preferably with just one sensor cable, rather than a dual cable for size constraint reasons. The length of the scintillating fiber can be adjusted for the needs of the treatment. For example, a length of about 2-4 cm or 3 cm may be suitable for monitoring dosage along the entire prostate. However, a smaller fiber of 0.5-1 cm will provide a more localized measurement.

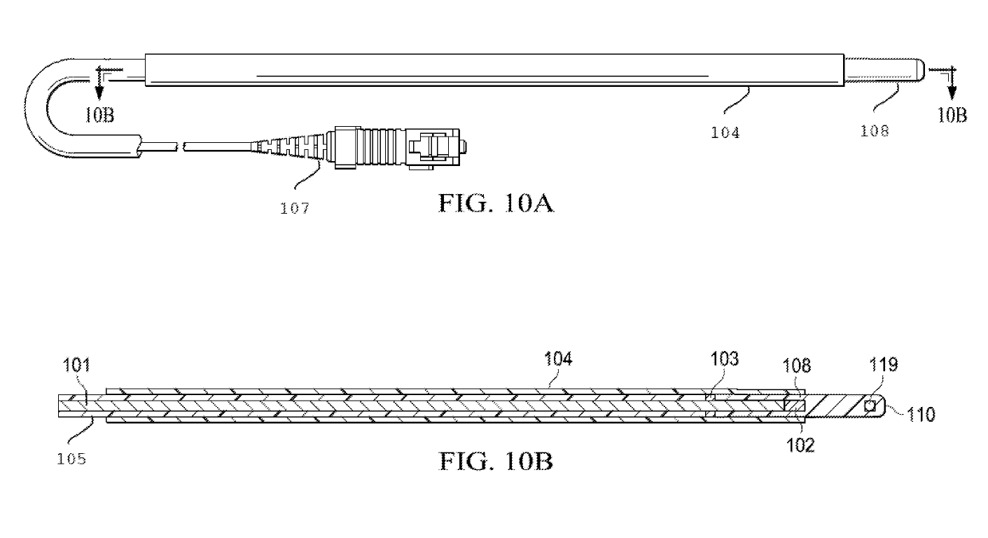

An exemplary simplex sensor cable having a single detector is shown in FIGS. 10A and 10B, wherein a fiber cap 108 has an embedded fiducial marker 119 in a distal tip thereof, and houses the scintillating fiber 102 and a portion of the optic fiber 101, partially stripped of its jacket 105 is directly abutted against the scintillating fiber 102 without glue therebetween. If desired, a drop of adhesive 103 can be used between the fiber cap 108 and the jacket 105, but this is optional. Preferably, a small piece of opaque heat shrinkable jacket 104 covers both the optic fiber 102/105 and fiber cap 108 and adds further protection to the sensor. The end of the cable is attached to adaptor 107.

In use, the distal tip of the catheter is usually coated with sterile lubricant, the catheter tip is inserted into the urinary meatus (orifice in a female) and the catheter gently fed into the urethra until 1-2 inches past where urine flow is noted. The position of the radiation sensor is ascertained by imaging the fiducial marker, and the position of the catheter and/or sensor adjusted so that the fiducial marker is positioned adjacent to the target treatment area. Preferably, the sensor is positioned to be adjacent the penile bulb, proximal to the prostate. In other instances, the sensor can be placed closer to the prostate.

If necessary, the catheter and/or cable can then be taped in position (e.g., on the patient's leg) to prevent accidental movement of the system. The cables are connected to a separate detector unit, e.g., photodetector. The urine lumen is connected to a collection bag, usually before insertion, and thus is available to collect urine throughout the procedure. Once the system is ready, radiation is applied to the target area, and the medical practitioner can thus monitor dosage in real time. When a target dosage level is reached, the practitioner can then cease the treatment at that target area or otherwise adjust treatment parameters. Balloon catheter use is similar, but includes the step of inflating the balloon portion of the catheter once the bladder has been reached, and deflating before removal.

In order to make assembly of the urinary catheter more efficient and reproducible, a special proximal end adaptor is made that provides the airtight connectors and bifurcation or trifurcation as needed. The simple tube of the catheter can be inserted into this hub, and the same hub used for e.g., urine drainage, inflation and cable entry.

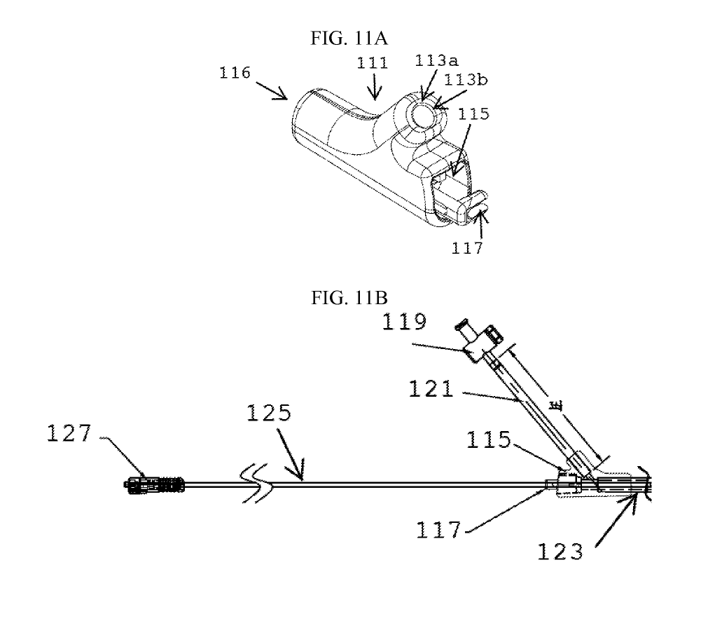

FIG. 11A-B shows one embodiment of a bifurcated hub lock 111, and the same principles can be applied to a trifurcated hub lock. The hub lock 111 fits over a proximal end of a catheter tube 123 and provides air and cable entry, but in an airtight manner, such that the cable 125 can enter tube or lumen 123 and so can air, from e.g., syringe 121, without leaking out. The hub lock 111 is bifurcated, having cable entry portal 115 as well air entry portal 113a, as well as lumen entry portal 116 opposite the bifurcated end. Preferably the various entry ports are all one way entry ports so that fluid cannot leak out. In the alternative, the two entry ports can connect with different lumens inside the tube 123, such that cable entry exit need not be airtight, although the catheter with balloon lumen is airtight. Of course, if the catheter is not a balloon catheter, the device need not be airtight at all, but most urinary catheters are balloon catheters.

Seal 113b shows e.g., an elastomeric seal 113b, but other one way valving is known and any method of providing an airtight seal or connection can be used, e.g., a one way valve or luer lock. The seal 113b thus will hold a suitable syringe 121, preventing air escape. Locking clip 117 positioned within cable entry portal 115 allows the user to close the clip, thus locking the sensor cable 125 to be locked into position, keeping it in one place during use. If desired, this clip 117 can provide an airtight cable entry point (not shown), but in other embodiments, a second elastomeric seal (not visible herein) or other valving can provide and airtight cable entry point. As yet another alternative, the cable entry portal 115 can connect only with the cable lumen not the air lumen, and thus this entry port need not be airtight.

In FIG. 11B, a bifurcated hub lock 111 is fit over the end of lumen 123, which inserts into portal 116. Syringe 121 fits into air portal 113a, providing an airtight fit by virtue of seal 113b. Cable 125 with terminal adaptor/connecter 127 is inserting through lock 117 and into cable entry portal 115. It is then locked into place by closing lock 117. As another option, the cable can be preassembled, but it is expected that the cable will be reusable, and the catheter itself disposable.

In use, the urinary catheter is assembled if needed by inserting the cable into the catheter via the hub lock, and feeding it up to near the target zone. The catheter with sensor cable is then inserted into the meatus or urethra and taped in place against the leg on reaching the bladder. The syringe is inserted into the hub lock, and the bladder balloon inflated, preventing egress of the catheter at the bladder end of the catheter. Drainage may be allowed throughout if needed, or if a separate draining lumen is not provided, urine can be drained with the syringe before inflation.

The sensor can then be imaged using the fiducial marker, adjusted as needed to e.g., target the prostate, and then the lock or clip 117 closed, preventing the sensor from moving with respect to the catheter. The adaptor or connector is connected to a separate reader device (not described herein), and radiation applied to e.g., the prostate. Dosage can thus be reliably obtained in real time using this active radiation sensor cable.

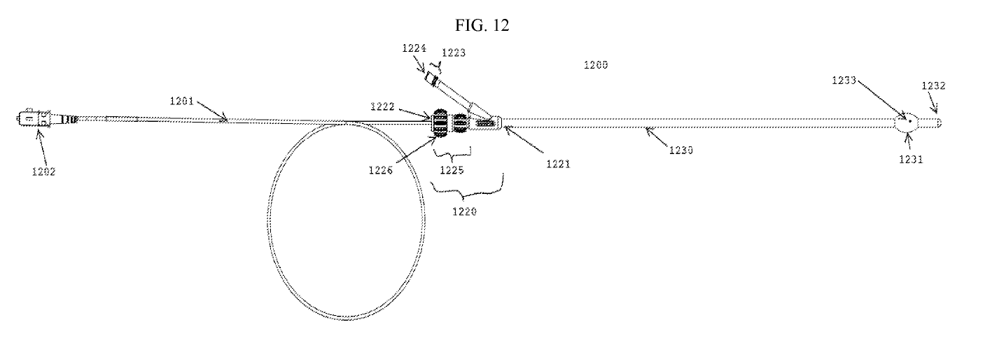

FIG. 12 shows yet another embodiment of the urinary catheter 1200 with radiation sensor cable 1201, having adaptor 1202 at the proximal end. The distal end of the radiation sensor is not visible, as being inside the catheter. Catheter tube 1230 has two lumens, one for air and the other for a sensor cable, although these are not visible in this exterior view. The bullet nose or full round (hemispherical) tip 1232 is closed, thus this embodiment does not allow for urine drainage, although this feature can be provided if desired. Balloon 1231 is shown inflated, and the hole 1233 in the air lumen is visible through the translucent balloon. When not inflated, the tiny balloon is flush with the exterior of the tube 1230, thus providing a smooth surface for entry into the urethra.

Locking hub 1220 has a bifurcated end opposite a single end. Single end has an entry port 1221 for airtight insertion of catheter tube 1230. Air entry portal 1224 is fitted e.g., with a spring valve connector 1223, for fluid tight connection to a syringe or other fluid source. A luer lock or other airtight connector could easily replace this spring valve connector. The cable entry port 1222 has a locking connector, in this case a Tuohy Borst connector 1225 (Merit Medical). The Tuohy Borst is a single-use, proprietary valved adapter that provides a leak-proof seal during interventional and diagnostic procedures. The cable can be inserted into the catheter through the connector 1225, and then the valve end 1226 rotated to close the connector down on the cable, thus both locking it in place and providing an airtight seal. Other connectors/locks could be used, however, and this is only one example of an off the self component that can be used, as the air tight feature is not essential in this embodiment. If both bifurcated ends provide airtight seals, then the interior of the hub lock need not have separate fluidic pathways for connection to the two lumens in the catheter tube.

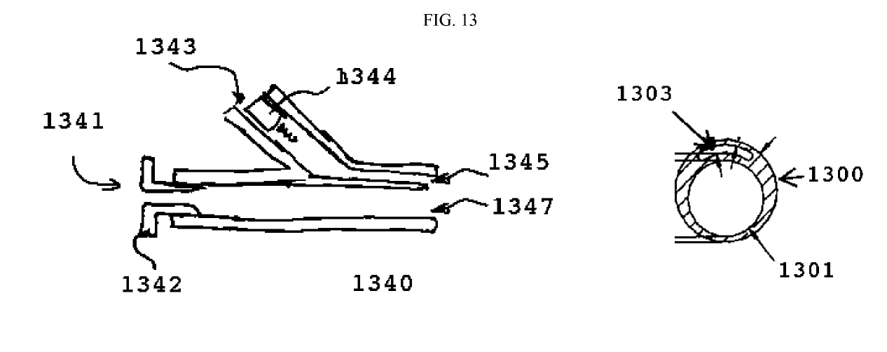

FIG. 13 shows another locking hub 1340 in cross section and catheter tube 1300 in cross section. The catheter tube fits into open single end, with fluidic pathway 1345 lining up with air lumen 1303. Cable inlet end 1347 lines up with cable lumen 1301. A spring valve 1344 in air inlet end 1343 allows airtight connection with a syringe or other means for providing fluid or air to the balloon. A luer lock would work equally well, as would an elastomeric seal. Cable entry end 1341 is fitted with a locking device 1342 for locking cable in position once the sensor tip reaches the target. Since there is a separate fluidic pathway inside the hub for inflation, this lock need not be airtight, although the redundancy provides backup in case the fluidic connection to the air lumen is not leak proof.

As above, in use the cable is inserted into the hub lock and fed to about an inch of so from the balloon (assuming the prostate is the target). The assembled urinary catheter with radiation sensor cable is then fed into the urethra to the bladder, then a fluid supply is connected to the fluid inlet end of the locking hub, and the balloon inflated, e.g., with sterile saline. Typically, the catheter is taped to the patient's leg, and the adapter plugged into a separate reader. The fiducial marker can then be imaged, and the sensor tip position adjusted as needed, and then again locked into place. This can be done before or after inflation, but preferably is after inflation so that the distal end of the catheter doesn't move. Radiation is applied, and the dosage read from the reader in real time, stopping the treatment when the final dosage level (for that session) is reached. The treatment can thus be monitored and dosage adjusted as needed during treatment.

In some embodiments, the method also includes transferring radiation dosage data to a patient medical record, preferably a non-transitory electronic medical record. In other embodiments, the record is provided as a printout.

In other embodiments, the method includes a non-transitory computer readable medium that stores thereon the medical record, and/or the software needed to analyze the raw data, correcting e.g., for temperature, background radiation, and being capable of providing real time dosage information.

The foregoing disclosure and description of the invention is illustrative and explanatory thereof. Various changes in the details of the illustrated construction can be made within the scope of the present claims without departing from the true spirit of the invention. The present invention should only be limited by the following claims and their legal equivalents.

1. A method comprising:
inserting a catheter into a urethra of a patient, the catheter comprising a catheter distal end, a catheter proximal end, and at least one lumen extending therebetween;
advancing the catheter along the urethra until the distal end of the catheter has been positioned a selected distance distally beyond a target treatment area; and
advancing at least one sensor along at least one lumen of the catheter, such that the at least one sensor is placed at the target treatment area; wherein the at least one sensor is comprised of a radiation sensor.

2. The method of claim 1, wherein after the catheter has been positioned the selected distance distally beyond the target treatment area, further comprising the step of imaging the catheter.

3. The method of claim 1, wherein the target treatment area is a location substantially adjacent to a penile bulb of the patient.

4. The method of claim 1, wherein the target treatment area is a location substantially adjacent to a prostate of the patient.

5. The method of claim 1, further comprising the step of:
connecting the at least one sensor to a photodetector.

6. The method of claim 5, further comprising the steps of:
delivering radiation to the target treatment area; and
monitoring in real time the dosage of delivered radiation.

7. The method of claim 6, further comprising the step of:
stopping delivery of radiation to the target treatment area upon a target dosage level of radiation being detected by the at least one sensor.

8. The method of claim 7, further comprising the step of:
transferring the monitored dosage of delivered radiation to a patient medical record.

9. The method of claim 6, further comprising the step of:
sensing the amount of delivered radiation with the at least one sensor.

10. The method of claim 6, wherein the monitored dosage of delivered radiation is stored on a computer readable medium.

11. A method comprising:
inserting a catheter into a urethra of a patient, the catheter comprising a catheter distal end, a catheter proximal end, and at least one lumen extending therebetween;
advancing the catheter along the urethra until the distal end of the catheter has been positioned a selected distance distally beyond a target treatment area;
advancing a sensor cable along at least one lumen of the catheter such that the sensor cable is placed at the target treatment area;
delivering radiation to the target treatment area;
monitoring in real time the dosage of delivered radiation; and
stopping delivery of radiation to the target treatment area upon a target dosage level of radiation being detected.

12. The method of claim 11, wherein the sensor cable is comprised of at least two radiation detecting sensors.

13. The method of claim 12, wherein the two radiation detecting sensors are configured to be in an offset position relative to each other.

14. The method of claim 13, wherein the catheter further comprises a balloon along the catheter distal end, and further comprising the step of:
advancing the catheter along the urethra until the balloon is placed within a bladder; and
inflating the balloon after it has been placed in the bladder, thereby securing the catheter in place.

15. A method comprising:
inserting a catheter into a urethra of a patient, the catheter comprising a catheter distal end, a catheter proximal end, and at least one lumen extending therebetween;
advancing the catheter into the urethra until urine flow is detected;
advancing a sensor cable along at least one lumen of the catheter to a target treatment area;
imaging the catheter after the sensor cable has been placed at the target treatment area; and
adjusting the position of the sensor cable within the at least one lumen of the catheter if the image shows the sensor cable is not correctly positioned relative to the target treatment area.

16. The method of claim 15, wherein the sensor cable is comprised of at least one radiation detecting sensor.

17. The method of claim 16, wherein the sensor cable is comprised of at least two radiation detecting sensors that are configured to be in an offset position relative to each other.

18. The method of claim 16, further comprising the steps of:
delivering radiation to the target treatment area; and
monitoring in real time the dosage of delivered radiation using the at least one radiation detecting sensor.

19. The method of claim 18, further comprising the steps of:
transferring the monitored dosage of delivered radiation to a patient medical record.

20. The method of claim 19, wherein the target treatment area is located a selected distance proximal to a prostate of the patient.
